(19) United States
(12) Patent Application Publication
(10) Pub. No.: US 2025/0123202 A1
(43) Pub. Date: Apr. 17, 2025

(54) METHOD AND DEVICE FOR DIFFUSE REFLECTANCE SPECTROSCOPY COMPRISING INTENSITY AND/OR OPTICAL FREQUENCY MODULATION OF THE OPTICAL RADIATION

(71) Applicant: COMMISSARIAT A L'ENERGIE ATOMIQUE ET AUX ENERGIES ALTERNATIVES, Paris (FR)

(72) Inventors: Luc ANDRE, Grenoble (FR); Anne KOENIG, Grenoble (FR); Mélina BARDET, Grenoble (FR)

(73) Assignee: COMMISSARIAT A L'ENERGIE ATOMIQUE ET AUX ENERGIES ALTERNATIVES, Paris (FR)

(21) Appl. No.: 18/909,494

(22) Filed: Oct. 8, 2024

(30) Foreign Application Priority Data
Oct. 17, 2023 (FR) 2311219

(51) Int. Cl. G01N 21/47

(52) U.S. Cl. CPC G01N 21/474; G01N 2201/0691; G01N 2201/1245

(57) ABSTRACT

A method for measuring the attenuation coefficient of a scattering and/or absorbing part of a body using diffuse reflectance spectroscopy. The method includes emitting optical radiation, the intensity and/or the optical frequency of which are modulated, with at least part of the optical radiation, called a probe signal, irradiating the body; receiving part of the probe signal, called a backscattered signal, that is scattered and reflected by the body and measuring the path length of the backscattered signal; measuring the reflectance of the part of the body traversed by the backscattered signal; and computing the attenuation coefficient based on the measured path length and on the reflectance.

### TECHNICAL FIELD

The present invention relates to the field of optical sensors for Diffuse Reflectance Spectroscopy, also referred to using the abbreviation “DRS”.

### PRIOR ART

Diffuse reflectance spectroscopy (DRS) is a non-invasive measurement technique for studying the structure and/or the composition of a scattering body. It is notably used to measure the concentration of chromophores in biological tissue, for example, to measure the rate of oxygenation and/or the rate of hydration and/or glycaemia in skin tissue.

FIG. 1 shows an example of the implementation of DRS according to the prior art for studying skin tissue 1. The skin tissue 1 comprises an epidermis 2 and a dermis 3 covered by the epidermis 2. The epidermis 2 comprises epidermal cells 4 and the dermis 3 comprises dermal cells 5 and blood vessels 6. The skin tissue 1 also comprises a corneous layer 7 made up of dead cells covering the epidermis 2.

A device for implementing DRS generally uses a light source 8 emitting optical radiation into the skin tissue 1 with a wavelength ranging between 400 nm and 1,700 nm. It further comprises optical sensors 9₁, 9₂ for detecting a backscattered part 10₁, or 10₂, of the optical radiation that has interacted with the skin tissue 1.

As they travel through the skin tissue 1, the photons of the optical radiation are absorbed, deflected and/or scattered by the absorbing or scattering constituents 4-6 of the skin tissue 1. Absorbing constituents 4-6 notably include chromophores, for example, water, some lipids, melanin, hemoglobin or glucose. Scattering constituents 4-6 notably include melanosomes, blood cells, collagen, keratin and some lipids. Only part 10₁, 10₂ of the photons is backscattered, i.e., it emerges by diffuse reflection from the side of the skin tissue 1 through which the optical radiation was introduced, and thus can be detected by one of the optical sensors 9₁, 9₂.

The optical sensor 9₁ measures the backscattered part 10₁ of the optical radiation in order to study the structure and/or the composition of the epidermis 2. The optical sensor 9₂ measures the backscattered part 10₂ of the optical radiation in order to study the structure and/or the composition of the dermis 3. As illustrated in FIG. 1, the distance between the sensors 9₁, 9₂ and the light source 8 is selected as a function of the depth, in the skin tissue 1, of the considered study zone. Similarly, the properties of the optical radiation, notably its wavelength and its angle of incidence on the skin tissue 1, are selected as a function of the constituents 4-6 of the skin tissue 1 to be studied.

Most current methods and devices for diffuse reflectance spectroscopy only implement optical radiation with a constant intensity. However, as discussed in the article by Hasan Ayaz et al., [1], the use of intensity-modulated optical radiation would theoretically allow more information to be more accurately obtained from diffuse reflectance spectroscopy.

However, the inventors are unaware of an existing device and/or method in the prior art for diffuse reflectance spectroscopy implementing modulated optical radiation and exhibiting better performance capabilities than the methods and devices for diffuse reflectance spectroscopy only implementing optical radiation that is not modulated over time.

Therefore, a requirement exists for a method and a device exhibiting high accuracy for measuring the attenuation of optical radiation backscattered by a scattering and/or absorbing body during the diffuse reflectance spectroscopy thereof.

### DISCLOSURE OF THE INVENTION

The invention relates to a method for measuring the attenuation coefficient of a scattering and/or absorbing part of a body using diffuse reflectance spectroscopy, the method comprising the following steps:
a) emitting optical radiation, the intensity and/or the optical frequency of which are modulated, with at least part of the optical radiation, called probe signal, irradiating the body;
b) receiving part of the probe signal, called backscattered signal, that is scattered and reflected by the body and measuring the path length d of the backscattered signal;
c) measuring the reflectance R of the part of the body traversed by the backscattered signal;
d) computing the attenuation coefficient μ based on the measured path length d and on the reflectance R.

The invention implements optical radiation that is intensity-modulated and/or optical frequency-modulated for diffuse reflectance spectroscopy in order to accurately measure the path length d of the backscattered signal. The invention therefore differs from the diffuse reflectance spectroscopy methods that are known in the prior art, which are not suitable for measuring the path length of a backscattered signal. In the prior art methods known to the inventors, the path length of a backscattered signal is estimated by digital simulation without measuring an optical signal. In these known methods of the prior art, measuring the attenuation coefficient suffers as a result of the approximate estimation of the path length of the backscattered signal, notably when the body is a scattering body.

The inventors have thus succeeded in proposing a method implementing diffuse reflectance spectroscopy measuring using intensity-modulated and/or optical frequency-modulated radiation with performance capabilities that are at least as good as those of the known methods that implement constant intensity optical radiation. Modulating the optical radiation allows the path length d to be measured without complexifying the implementation of the method.

Advantageously, the body whose attenuation coefficient is measured by the method according to the invention is a scattering, homogeneous or heterogeneous body. Finally, the present invention is easy to implement in practice.

The “path length d of the backscattered signal” is the average length of the optical paths covered by the backscattered signal.

The scattering medium can be solid or fluid, notably liquid and/or gaseous. It can comprise solid elements dispersed in a fluid. It can comprise droplets of a liquid suspended in a gas.

The scattering medium can be biological tissue. The biological tissue notably can be part of an animal body, in particular a human body, for example, skin. By way of a variant, the biological tissue can be plant tissue.

Preferably, the optical radiation is laser.

The wavelength of the optical radiation is selected as a function of the absorption spectrum of the one or more elements contained in the body intended to be observed using diffuse reflectance spectroscopy. Preferably, the optical radiation has a wavelength ranging between 400 nm and 1,700 nm.

The attenuation coefficient μ can be computed based on the following formula, derived from Beer-Lambert's law:

$$R = \frac{{e}^{- μ d}}{{d}^{2}} . \qquad [ Math . 1 ]$$

The formula [Math 1] is an initial approximation of the attenuation coefficient μ. Other formulae can be used, notably originating from a more precise physical model, for example, taking into account the extinction phenomenon.

Alternatively, the attenuation coefficient μ can be divided into an absorption coefficient μₐ and a diffusion coefficient μₛ, and the attenuation coefficient μ can be computed based on the following formula:

$$R = \frac{{z}_{0}}{4 π} \cdot [ ( {μ}_{e f f} + \frac{1}{{r}_{1}} ) \frac{{e}^{- {μ}_{e f f} \cdot {r}_{1}}}{{r}_{1}^{2}} + ( 1 + \frac{4}{3} A ) ( {μ}_{e f f} + \frac{1}{{r}_{2}} ) \frac{{e}^{- {μ}_{e f f} \cdot {r}_{2}}}{{r}_{2}^{2}} ] \qquad [ Math . 2 ]$$

with

$${z}_{0} = \frac{1}{{μ}_{a} + {μ}_{s} ( 1 - g )}$$

$${μ}_{e f f} = \sqrt{3 {μ}_{a} ( {μ}_{a} + {μ}_{s} ( 1 - g ) )}$$

where g is the anisotropy factor,

$$A = \frac{1 + {r}_{i}}{1 - {r}_{i}}$$

$${r}_{i} = - 1.440 {n}^{- 2} + 0.710 {n}^{- 1} + 0.0636 n + 0.668$$

where n equals the ratio of the optical index of the part of the body traversed by the backscattered signal to the optical index of the environment outside the body;
with r₁ and r₂ being factors depending on the path length d and the part of the body traversed by the backscattered signal.

The equation [Math 2] can be deduced based on the diffusion equation provided in the article by Farrell T. J., et al.: “A diffusion theory model of spatially resolved, steady-state diffuse reflectance for the noninvasive determination of tissue optical properties in vivo”, Med. Phys., 1992, 19 (4), 879-88.

It is also possible to use a Monte-Carlo type model to compute the attenuation coefficient μ, notably in the event that the approximation of the diffusion is no longer valid, i.e., the absorption coefficient μₐ is negligible compared to the reduced diffusion coefficient μₛ′ and the distance L between the point where the probe signal enters the body and the point where the backscattered signal emerges from the body is very small compared to the inverse of the reduced diffusion coefficient μₛ′. The reduced diffusion coefficient μₛ′ is equal to μₛ (1−g), where g is the anisotropy factor. The use of a Monte-Carlo type model allows the trajectory of the photons to be simulated by using random methods. Each photon is considered to be an individual particle whose path and interactions are monitored. A statistical result is then obtained by sending a very large number of photons, typically of the order of 10⁶ photons.

Measuring the reflectance R can involve measuring the average intensity iᵣ of the backscattered signal and computing the reflectance R using the following formula:

$$R = \frac{{i}_{r}}{{i}_{e}} \qquad [ Math 3 ]$$

where iₑ is the average intensity of the probe signal irradiating the body.

The average intensity of an optical signal is the average value of the intensity of said optical signal integrated over a modulation period of said optical signal.

As a variant, measuring the reflectance R comprises transmitting an additional probe signal having the same wavelength as the optical radiation and irradiating the body, followed by measuring the average intensity iᵣ′ of part of the additional probe signal, called additional backscattered signal, scattered and reflected by the body and then computing the reflectance R using the following formula:

$$R = \frac{{i}_{r}^{′}}{{i}_{e}^{′}} \qquad [ Math 4 ]$$

where iₑ′ is the average intensity of the additional probe signal irradiating the body.

Preferably, the additional probe signal has a constant intensity.

According to another variant, step a) comprises modulating the optical frequency of the optical radiation over a ramp of height h and duration t, and comprises splitting the optical radiation into the probe signal and into a local oscillator, with the local oscillator not irradiating the body,

the measurement of the reflectance R comprising mixing the backscattered signal and the local oscillator in order to form an electromagnetic beat, followed by computing the reflectance R based on the modulation amplitude of the intensity of the electromagnetic beat and/or based on the value of the direct component of the intensity of the electromagnetic beat. The reflectance R can be obtained based on the amplitude of the Fourier transform of the intensity of the electromagnetic beat.

Preferably, the intensity of the optical radiation is sinusoidally modulated.

Preferably, the modulation frequency of the intensity of the optical radiation ranges between 10 and 1,000 MHz. Advantageously, the higher the modulation frequency, the better the measurement accuracy.

Preferably, the average intensity iᵣ of the backscattered signal or the average intensity iᵣ′ of the additional backscattered signal is measured by a current-assisted photonic demodulator or by a PIN junction photodiode.

According to a first embodiment, the intensity of the optical radiation can be sinusoidally modulated at a modulation frequency f_{mod}, the measurement of the path length d can comprise measuring the phase shift α between the backscattered signal and the emitted optical radiation, followed by computing the path length d based on the following formula:

$$d = c \cdot \frac{α}{4 \cdot π \cdot {f}_{m o d}} \qquad [ Math 5 ]$$

where c is the speed of light in a vacuum.

Preferably, the phase shift α is measured by a current-assisted photonic demodulator.

According to a second embodiment, to measure the path length d, step a) comprises modulating the optical frequency of the optical radiation over a ramp of height h and duration t; and
step a) comprises splitting the optical radiation into the probe signal and into a local oscillator, with the local oscillator not irradiating the body;
step b) comprises mixing the backscattered signal and the local oscillator in order to form an electromagnetic beat, followed by computing the path length d based on the following formula:

$${f}_{beat} = h \cdot \frac{d}{c \cdot t} \qquad [ Math 6 ]$$

where f_{beat} is the average frequency of the electromagnetic beat and c is the speed of light in a vacuum.

Preferably, the height h ranges between 12 and 40 GHz.

Preferably, the duration t ranges between 0.1 and 10 ms.

Preferably, the optical radiation is split so that the intensity of the local oscillator during the mixing forming the electromagnetic beat is substantially equal to the intensity of the backscattered signal during said mixing. Advantageously, the sensitivity of the diffuse reflectance spectroscopy is then maximized.

Obtaining the frequency f_{beat} of the electronic beat can involve measuring the intensity of the electromagnetic beat over time. Preferably, the intensity of the electromagnetic beat is measured by a PIN junction photodiode, preferably two PIN junction photodiodes electrically connected in series. The one or more PIN junction photodiodes can be made of germanium, silicon or indium-gallium arsenide. Silicon is preferred for optical radiation with wavelengths ranging between 400 and 940 nm. Indium-gallium arsenide is preferred for optical radiation with wavelengths ranging between 1 and 2.5 μm.

Preferably, the frequency f_{beat} of the electromagnetic beat is obtained by processing involving a Fourier transform of the intensity of the electromagnetic beat.

The present invention also relates to a device for implementing the method according to the invention according to the first embodiment, the device comprising:
a light emission source configured to emit sinusoidally intensity-modulated optical radiation, preferably with a wavelength ranging between 400 nm and 1,700 nm, and at least part of which, called probe signal, is intended to irradiate a scattering and/or absorbing body;
an optical detector configured to measure the phase shift α between part of the probe signal, called backscattered signal, scattered and reflected by the body and the emitted optical radiation, and to measure the average intensity of the backscattered signal, with the light emission source and the optical detector being designed to be disposed on the same side of the body;
a data processing unit configured to compute the path length d based on the formula [Math 5], and to compute the attenuation coefficient μ based on the computed path length d and on the reflectance R of the part of the body traversed by the backscattered signal measured based on the average intensity measured by the optical detector.

Preferably, the optical detector comprises at least one current-assisted photonic demodulator, preferably a plurality of current-assisted photonic demodulators. Preferably, the current-assisted photonic demodulators are regularly arranged in a plane.

Advantageously, a current-assisted photonic demodulator is able to measure a phase shift between the backscattered signal and the emitted optical radiation by receiving only the backscattered signal. To this end, the current-assisted photonic demodulator can be calibrated with a detection frequency that is a multiple of the intensity-modulation frequency of the optical radiation. It is also able to measure the intensity of an optical signal over time. Thus, if said optical signal has a constant intensity, then the measured intensity is equal to its average intensity. Alternatively, if said optical signal is intensity-modulated, then the average intensity measured by the photonic demodulator can be determined by integrating the measured intensity over a complete modulation period.

Preferably, the optical detector comprises at least one PIN junction photodiode, preferably a plurality of PIN junction photodiodes. Preferably, the PIN junction photodiodes are regularly arranged in a plane. The one or more PIN junction photodiodes can be made of germanium, silicon or indium-gallium arsenide.

Preferably, the distance between the one, or at least one, preferably each, of the PIN junction photodiodes and the nearest current-assisted photonic demodulator is less than 100 μm, preferably less than 10 μm. Thus, the photons collected by said current-assisted photonic demodulator and by said PIN junction photodiode have substantially the same path length and have traversed the same part of the scattering and/or absorbing body.

Preferably, the photonic demodulators are alternated with the PIN junction photodiodes. For example, the photonic demodulators can be alternated in a staggered manner with the PIN junction photodiodes. Notably, the photonic demodulators and the junction photodiodes can be arranged as a checkerboard pattern. The photonic demodulators also can be alternated with the PIN junction photodiodes so that each photonic demodulator is surrounded by eight adjacent PIN junction photodiodes.

A plurality of current-assisted photonic demodulators and/or a plurality of PIN junction photodiodes advantageously allows several backscattered optical signals to be collected that originate from the same probe signal, each of which has traversed different parts of the scattering and/or absorbing body. Thus, this allows the path length and the attenuation coefficient for different parts of the same scattering and/or absorbing body to be measured quickly and easily, which is particularly useful when the body is heterogeneous and/or stratified.

The invention also relates to a device for implementing the method according to the invention according to the second embodiment, the device comprising:
a light emission source configured to emit coherent optical radiation, preferably with a wavelength ranging between 400 nm and 1,700 nm, with an optical frequency that is modulated over a ramp of height h and duration t;
a beam splitter configured to split the optical radiation into a probe signal for irradiating a scattering and/or absorbing body and at least one local oscillator;
an optical detection unit comprising at least one multiplexer for mixing the local oscillator with part of the probe signal, called backscattered signal, scattered and reflected by the body in order to form an electromagnetic beat, with the optical detection unit further comprising at least one optical receiver configured to measure the intensity of the electromagnetic beat over time and to measure the average intensity of the backscattered signal, with the light emission source, the beam splitter and the optical detection unit being designed to be disposed on the same side of the body;
a data processing unit configured to determine the average frequency f_{beat} of the electromagnetic beat based on the measured intensity of said electromagnetic beat and to compute the path length d based on the formula [Math 6], and to compute the attenuation coefficient μ based on the computed path length d and on the reflectance R of the part of the body traversed by the backscattered signal measured based on the average intensity of the backscattered signal measured by the optical receiver or based on the intensity of the electromagnetic beat.

Preferably, the beam splitter is configured so that the phase and the fundamental frequency of the local oscillator and the phase and the fundamental frequency of the probe signal are respectively equal.

Preferably, the optical detection unit is configured to separately measure the direct component of the intensity of the electromagnetic beat and the alternating component of the intensity of the electromagnetic beat. Thus, the reflectance R can be computed based on the modulation amplitude of the alternating component and/or based on the value of the direct component. This separation also simplifies the determination of the average frequency f_{beat}.

Preferably, the optical detection unit comprises a plurality of optical receivers and as many multiplexers as optical receivers, the beam splitter is configured to split the optical radiation into a probe signal and as many local oscillators as optical receivers, each of the multiplexers is configured to mix one of the local oscillators with a backscattered signal, in order to form an electromagnetic beat directed toward one of the optical receivers. Advantageously, a plurality of optical receivers allows several backscattered optical signals to be collected that originate from the same probe signal, each of which has traversed a different part of the scattering and/or absorbing body. Thus, this allows the path length and the attenuation coefficient for different parts of the same scattering and/or absorbing body to be measured quickly and easily, which is particularly useful when the body is heterogeneous and/or stratified.

Preferably, the device comprises at least one waveguide for guiding the local oscillator from the beam splitter to the optical detection unit. The waveguide can be made of a material selected from silicon, silicon nitride, silicon dioxide and mixtures thereof. The waveguide can be an optical fiber.

Preferably, the optical receiver comprises at least one PIN junction photodiode, preferably two PIN junction photodiodes electrically connected in series. The one or more PIN junction photodiodes can be made of germanium, silicon or indium-gallium arsenide. Preferably, the optical receiver comprises an optical amplifier for amplifying the difference in electrical current between the two PIN junction photodiodes.

Preferably, the data processing unit is configured so that the attenuation coefficient μ is computed based on the formula [Math 1] or [Math 2].

Preferably, the light emission source is configured to emit an additional probe signal with the same wavelength as the optical radiation, with a constant intensity and that is intended to irradiate the body.

Further advantages and features will become more clearly apparent upon reading the detailed description, which is illustrative and non-limiting, and with reference to the following figures.

### BRIEF DESCRIPTION OF THE DRAWINGS

FIG. 1 is a schematic cross-sectional representation of an embodiment of a measurement using diffuse reflectance spectroscopy that is known in the prior art.

FIG. 2 is a schematic cross-sectional representation of an example of a measurement using diffuse reflectance spectroscopy according to the first embodiment of the method according to the invention.

FIG. 3 is a graph showing the evolution of the light intensity of the probe signal and of the backscattered signal over time when implementing the method illustrated in FIG. 2.

FIG. 4 is a schematic cross-sectional representation of an example of a measurement using diffuse reflectance spectroscopy according to the second embodiment of the method according to the invention.

FIG. 5 is a graph showing the evolution of the light intensity of the additional probe signal and of the additional backscattered signal over time.

FIG. 6 is a schematic top view representation of an optical detector of a device according to the invention, with the optical detector comprising a plurality of current-assisted photonic demodulators and a plurality of PIN junction photodiodes.

FIG. 7 is a schematic cross-sectional representation of an example of a device comprising the optical detector according to FIG. 6 for a measurement using diffuse reflectance spectroscopy.

FIG. 8 is a schematic top view representation of an optical detector of a device according to the invention, with the optical detector comprising a plurality of current-assisted photonic demodulators.

FIG. 9 is a schematic cross-sectional representation of a third example of a measurement using diffuse reflectance spectroscopy according to the invention.

FIG. 10 is a graph showing the evolution of the optical frequency of a probe signal over a ramp.

### DETAILED DESCRIPTION

In the figures, the various constituent elements of the device according to the invention and the scattering and/or absorbing medium have not necessarily been shown to scale, for the sake of the clarity of the drawing.

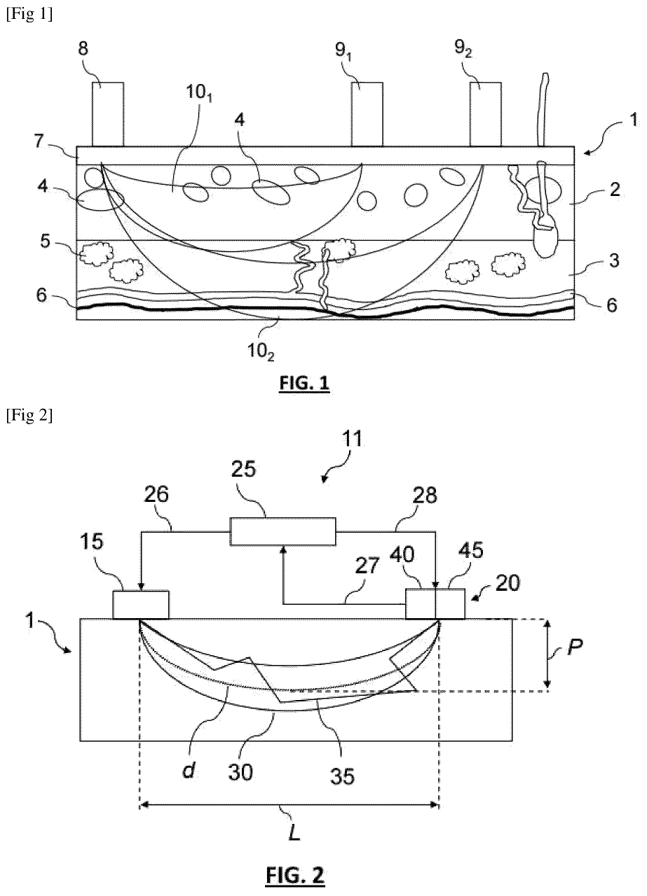

FIG. 1 has been described above.

FIG. 2 illustrates an embodiment of a device 11 for diffuse reflectance spectroscopy of a body 1. The device 11 comprises a light emission source 15 and an optical detector 20, both in surface contact with the scattering and/or absorbing body 1. The device 11 also comprises a data processing unit 25 connected to the light emission source 15 and the optical detector 20.

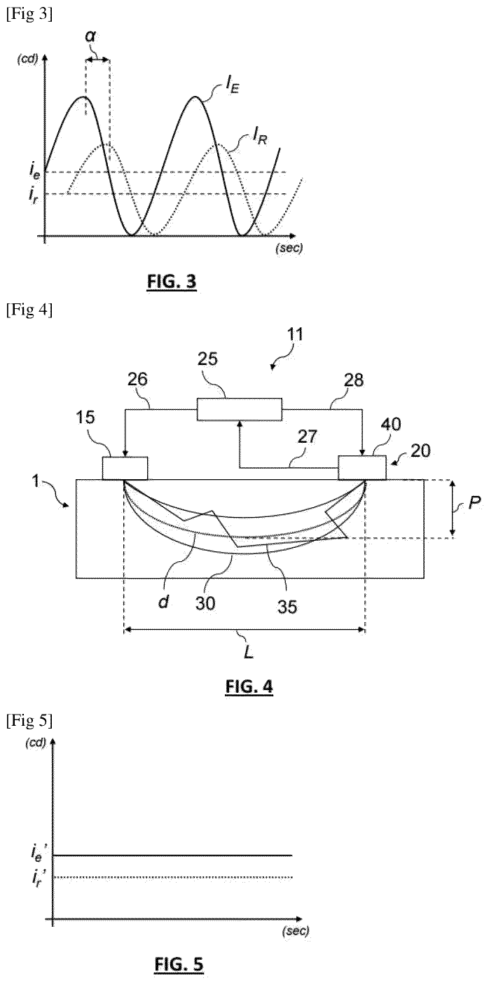

Under instructions 26 from the data processing unit 25, the light emission source 15 emits intensity-modulated optical radiation with a wavelength ranging between 400 nm and 1,700 nm. At least part, preferably all, of the optical radiation is directed so as to irradiate the body 1. This part is called the probe signal. FIG. 3 shows the evolution of the intensity of the probe signal as a function of time. The probe signal comprises a direct component with a constant intensity equal to i_{DC} and a sinusoidally intensity-modulated alternating component with an amplitude equal to i_{AC} and a frequency equal to f_{mod}. Furthermore, the average intensity iₑ of the probe signal over time is equal to i_{DC}. The evolution of the intensity I_{E} of the probe signal over time is provided by the following formula:

$${I}_{E} ( t ) = {i}_{D C} + {i}_{A C} \cdot sin ( 2 π {f}_{m o d} t ) . \qquad [ Math 7 ]$$

As it travels through the body 1, the probe signal is partly scattered and reflected by the body 1. The optical detector 20 receives and collects part of the probe signal, called backscattered signal, scattered and reflected by the body 1. FIG. 3 shows the evolution of the intensity of the backscattered signal as a function of time. The backscattered signal comprises a direct component with a constant intensity equal to R.i_{DC} and a sinusoidally intensity-modulated alternating component with an amplitude equal to R.i_{AC} and a frequency equal to f_{mod}, where R is the reflectance of the part of the body 1 traversed by the backscattered signal. The backscattered signal has a phase difference α with the optical radiation emitted by the light emission source 15. The average intensity iᵣ of the backscattered signal over time is equal to R.i_{DC}. The evolution of the intensity I_{R} of the backscattered signal over time is provided by the following formula:

$${I}_{R} ( t ) = R \cdot {i}_{D C} + R \cdot {i}_{A C} \cdot sin ( 2 π {f}_{m o d} t + α ) . \qquad [ Math 8 ]$$

FIG. 2 illustrates the envelope 30 of the backscattered signal. The envelope 30 contains the statistical set of the trajectories 35 of the photons received and collected by the optical detector 20. The backscattered signal has a path length d, corresponding to the average length of the trajectories 35 of the photons received and collected by the optical detector 20. The backscattered signal also has a depth P corresponding to the average depth reached by the photons received and collected by the optical detector 20. This depth P depends on the distance L between the point where the probe signal enters the body 1 and the point where the backscattered signal emerges from the body, the greater this distance L, the greater the depth P.

In the embodiment illustrated in FIG. 2, the optical detector 20 comprises a Current-Assisted Photonic Demodulator (CAPD) 40 and a PIN junction photodiode 45. The current-assisted photonic demodulator 40 is attached to the PIN junction photodiode 45. The current-assisted photonic demodulator 40 is configured to measure the phase shift α between the backscattered signal and the optical radiation emitted by the light emission source 15. The PIN junction photodiode 45 is configured to measure the average intensity iᵣ of the backscattered signal over time. Once the phase shift α and the average intensity iᵣ have been measured, the optical detector 20 transmits them to the data processing unit 25 via the link 27. If required, the data processing unit 25 can control the optical detector 25 via the link 28, for example, in order to activate/deactivate the optical detector 25 and/or to calibrate the current-assisted photonic demodulator 40, notably its detection frequency.

After receiving the measured phase shift α and average intensity iᵣ, the data processing unit 25 computes the path length d based on the previously provided formula [Math 5] and computes the reflectance R equal to the ratio of the average intensity iᵣ of the backscattered signal to the average intensity iₑ of the probe signal. The data processing unit 25 then computes the attenuation coefficient μ based on the Beer-Lambert's formula [Math 1] or the formula [Math 2].

FIG. 4 illustrates another embodiment of a device 11 according to the invention for diffuse reflectance spectroscopy of a body 1. According to this embodiment, the optical detector 20 can be devoid of a PIN junction photodiode 45. Similar to the embodiment illustrated in FIG. 2, the current-assisted photonic demodulator 40 measures the phase shift α between the backscattered signal and the optical radiation emitted by the light emission source 15 and the data processing unit 25 computes the path length d of the backscattered signal based on the previously provided formula [Math 5].

The method implementing the device 11 illustrated in FIG. 4 differs from that illustrated in FIG. 2 in that the reflectance R of the part of the body 1 traversed by the backscattered signal is measured. For this measurement, the light emission source 15 emits an additional probe signal toward the body 1 that is the same wavelength as the optical radiation. The additional probe signal has a constant intensity iₑ′ over time, as shown in FIG. 5. For this measurement of the reflectance R, the positions of the light emission source 15 and of the optical detector 20 are the same as for the emission of the optical radiation and the reception of the probe signal.

As it travels through the body 1, the additional probe signal is partly scattered and reflected by the body 1. The optical detector 20 receives and collects part of the additional probe signal, called additional backscattered signal, scattered and reflected by the body 1. The additional backscattered signal has a constant intensity iᵣ′ over time equal to R.iₑ′, where R is the reflectance of the part of the body 1 traversed by the additional backscattered signal. The trajectories 35 of the photons of the additional backscattered signal received and collected by the optical detector 20 are similar to the trajectories 35 of the photons of the backscattered signal. Also, the additional backscattered signal has the same envelope 30, the same path length d and the same depth P as the backscattered signal.

In the embodiment illustrated in FIG. 4, the current-assisted photonic demodulator 40 measures the intensity iᵣ′ of the additional backscattered signal. Then, the data processing unit 25 computes the reflectance R that is equal to the ratio of the intensity iᵣ′ of the additional backscattered signal to the intensity iₑ′ of the additional probe signal. The data processing unit 25 then computes the attenuation coefficient μ based on, for example, the formula [Math 1] derived from the Beer-Lambert's law or the formula [Math 2].

The measurement of the reflectance R by transmitting an additional probe signal can be carried out before or after the measurement of the phase shift α between the backscattered signal and the optical radiation, as it is independent thereof.

In the embodiments shown in FIGS. 2 and 4, the optical detector 20 comprises only a single current-assisted photonic demodulator 40, and, where applicable, a single PIN junction photodiode 45. Furthermore, these embodiments are not suitable for simultaneously measuring the attenuation coefficient μ of different parts of the body 1. They are suitable for measuring the coefficient of only a single part of the body 1, with said part corresponding to the envelope 30 of the collected backscattered signal.

According to other embodiments, the optical detector 20 can comprise a plurality of current-assisted photonic demodulators 40, and where applicable, a plurality of PIN junction photodiodes 45.

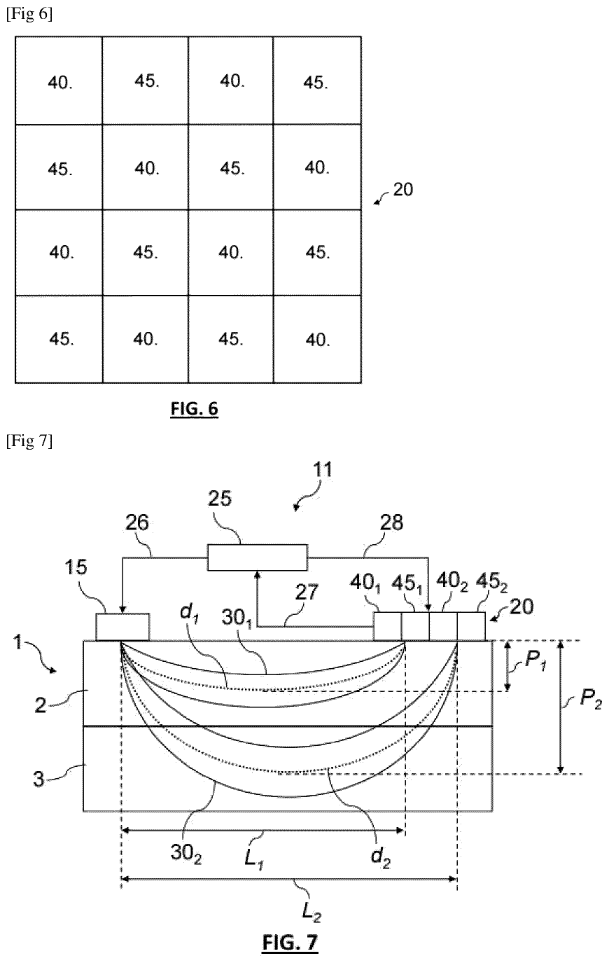

For example, as illustrated in FIG. 6, the optical detector 20 can comprise a plurality of current-assisted photonic demodulators 40 and a plurality of PIN junction photodiodes 45. The current-assisted photonic demodulators 40 are attached to the PIN junction photodiodes 45 in a staggered manner in a plane. This optical detector 20 is suitable for simultaneously measuring the attenuation coefficient μ of different parts of the body 1.

FIG. 7 illustrates an operating mode of a device 11 according to the invention comprising the optical detector 20 illustrated in FIG. 6. Similar to the mode illustrated in FIG. 2, the light emission source 15 emits optical radiation, part of which, called probe signal, illuminates the body 1. A first part of the probe signal, called first backscattered signal, scattered and reflected by the body 1 is received by a first current-assisted photonic demodulator 40₁ and a first PIN junction photodiode 45₁. A second part of the probe signal, called second backscattered signal, scattered and reflected by the body 1 is received by a second current-assisted photonic demodulator 40₂ and a second PIN junction photodiode 45₂.

The first backscattered signal has a different envelope 30₁ from the envelope 30₂ of the second backscattered signal. Notably, the depth P₁ of the first backscattered signal is less than the depth P₂ of the second backscattered signal. This is due to the distances L₁, respectively L₂, between the point where the probe signal enters the body 1 and the point where the first backscattered signal, respectively the second backscattered signal, emerges from the body 1. In other words, the envelopes 30₁ and 30₂ depend on the positioning of the current-assisted photonic demodulators 40₁ and 40₂ and of the PIN junction photodiodes 45₁ and 45₂ relative to the light emission source 15. Thus, the part of the body 1, called first part, traversed by the first backscattered signal differs from the part of the body 1, called second part, traversed by the second backscattered signal.

The path length d₁ of the first backscattered signal and the attenuation coefficient μ₁ of the first part are obtained in a similar manner to the example illustrated in FIG. 2, as are the path length d₂ of the second backscattered signal and the attenuation coefficient μ₂ of the second part.

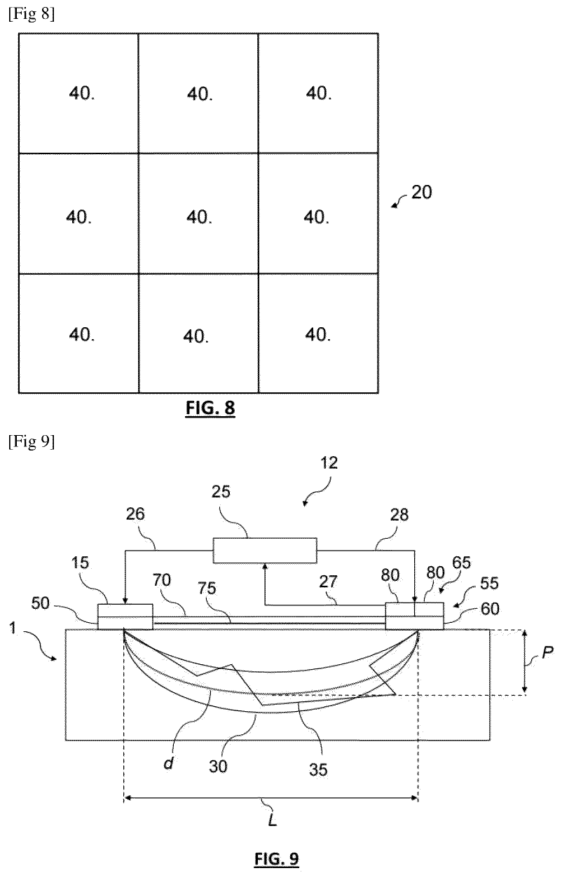

According to another example illustrated in FIG. 8, the optical detector 20 can comprise a plurality of current-assisted photonic demodulators 40 attached to each other in a plane. Each current-assisted photonic demodulator 40 is configured to receive a backscattered signal in a similar manner to the embodiment illustrated in FIG. 4, with each of said backscattered signals having traversed part of the body 1 distinct from the parts traversed by the other backscattered signals. Thus, the optical detector 20 allows an attenuation coefficient μ to be measured for each part of the body 1 traversed by the backscattered signals received by the current-assisted photonic demodulators 40.

FIG. 9 illustrates an example of a device 12 according to the invention for diffuse reflectance spectroscopy of a body 1. The device 12 comprises a light emission source 15, a beam splitter 50 and an optical detection unit 55, all arranged on the same side of the scattering and/or absorbing body 1. The device 12 also comprises a data processing unit 25 connected to the light emission source 15 and the optical detection unit 55.

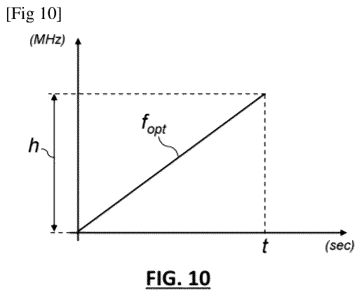

Under instructions 26 from the data processing unit 25, the light emission source 15 emits optical radiation with a wavelength ranging between 400 nm and 1,700 nm, the optical frequency of which is modulated. FIG. 10 illustrates the evolution of the optical frequency fₒₚₜ over time, which follows a ramp of height h and duration t.

The optical radiation is split by the beam splitter 50 into a probe signal that irradiates the body 1 and a local oscillator 75 that is directed by a waveguide 70 to the optical detection unit 55. The evolution of the intensity of the probe signal as a function of time is similar to that shown in FIG. 3. The probe signal comprises a direct component with a constant intensity equal to i_{DC} and a sinusoidally intensity-modulated alternating component, with an amplitude equal to i_{AC} and an optical frequency fₒₚₜ that is also modulated. Furthermore, the average intensity iₑ of the probe signal over time is equal to i_{DC}. The evolution of the intensity I_{E} of the probe signal over time is similar to the formula [Math 6]. The intensity of the local oscillator 75 evolves in a similar manner to the intensity I_{E} of the probe signal and differs only in terms of amplitude.

The probe signal enters the body 1 and is then partly scattered and reflected by the body 1. The optical detection unit 55 receives part of the probe signal, called backscattered signal, scattered and reflected by the body 1. The backscattered signal comprises a direct component with a constant intensity equal to R.i_{DC} and a sinusoidally intensity-modulated alternating component, with an amplitude equal to R.i_{AC} and an optical frequency fₒₚₜ that is also modulated, where R is the reflectance of the part of the body 1 traversed by the backscattered signal. The backscattered signal has a phase difference α with the local oscillator 75. The phase difference α is characteristic of the additional time induced by the body 1 on the backscattered signal in order to reach the optical detection unit 55. The average intensity iᵣ of the backscattered signal over time is equal to R.i_{DC}. The evolution of the intensity I_{R} of the backscattered signal over time is provided by the following formula [Math 7].

The optical detection unit 55 comprises a multiplexer 60 that mixes the local oscillator 75 with the backscattered signal. As the local oscillator 75 and the backscattered signal are out of phase, their mixing forms an electromagnetic beat by interference.

The optical detection unit 55 also comprises an optical receiver 65 comprising two PIN junction photodiodes 80 in series. The PIN junction photodiodes 80 receive the electromagnetic beat and measure the evolution of its intensity over time. The optical receiver 65 transmits the evolution of the measured intensity to the data processing unit 25.

The data processing unit 25 determines the average frequency f_{beat} of the electromagnetic beat based on the Fourier transform of its measured intensity. As explained in patent application FR 2113799 A, the average frequency f_{beat} of the electromagnetic beat is characteristic of, notably proportional to, the average time taken for the photons of the probe signal to traverse the body 1. The average frequency f_{beat} is therefore also characteristic of the path length d of the backscattered signal. Thus, the data processing unit 25 computes the path length d based on the average frequency f_{beat} and on the previously provided formula [Math 5].

The device 12 also measures the reflectance R of the part of the body 1 traversed by the backscattered signal. For this measurement, the positions of the light emission source 15 and of the optical detection unit 55 are the same as for the emission of the optical radiation and the reception of the probe signal. This measurement involves the light emission source 15 transmitting an additional probe signal irradiating the body 1. Preferably, the additional probe signal has a constant intensity.

As it travels through the body 1, the additional probe signal is partly scattered and reflected by the body 1. The optical detection unit 55 receives and collects part of the additional probe signal, called additional backscattered signal, scattered and reflected by the body 1. The intensity of the additional backscattered signal is proportional to the intensity of the transmitted additional probe signal by a factor that is equal to the reflectance R. As explained above, the additional backscattered signal has the same envelope 30, the same path length d and the same depth P as the backscattered signal.

The PIN junction photodiodes 80 measure the intensity of the additional backscattered signal. The optical detection unit 55 transmits the measured intensity to the data processing unit 25, which then computes the reflectance R equal to the ratio of the intensity of the additional backscattered signal to the intensity of the additional probe signal. The data processing unit 25 then computes the attenuation coefficient μ based on the Beer-Lambert's formula [Math 1] or on the formula [Math 2].

Measuring reflectance R by transmitting an additional probe signal can be carried out before or after measuring the average frequency f_{beat} of the electromagnetic beat, since it is independent thereof.

As a variant, the reflectance R can be measured based on the modulation amplitude of the intensity of the electromagnetic beat and/or based on the value of the direct component of the intensity of the electromagnetic beat. Notably, the evolution of the intensity I_{beat} of the electromagnetic beat over time is provided by the following formula:

$${I}_{beat} ( t ) = {i}_{tot} + ( R - 1 ) \cdot {i}_{D C} + \sqrt{R \cdot {i}_{D C} \cdot ( {i}_{tot} - {i}_{D C} )} \cdot sin ( 2 π {f}_{beat} t + ϕ ) , \qquad [ Math 8 ]$$

where ϕ is any phase, iₜₒₜ is the average intensity of the optical radiation and i_{DC} is the average intensity of the probe signal.

Since iₜₒₜ and i_{DC} are constants, it can be seen that the modulation amplitude of the intensity of the electromagnetic beat is directly proportional to the square root of the reflectance R and that the direct component of the intensity of the electromagnetic beat is proportional to the reflectance R.

Other variants and improvements can be contemplated without departing from the scope of the invention as defined by the following claims.

### LIST OF CITED DOCUMENTS

[1] Hasan Ayaz et al.: “Optical imaging and spectroscopy for the study of the human brain: status report”, Neurophoton, Vol. 9, No. S2, S24001, 30 Aug. 2022, https://doi.org/10.1117/1.NPh.9.S2.S24001

1. A method for measuring an attenuation coefficient of a scattering and/or absorbing part of a body using diffuse reflectance spectroscopy, the method comprising the following steps:
(a) emitting optical radiation, an intensity and/or an optical frequency of which are modulated, with at least part of the optical radiation, called a probe signal, irradiating the body;
(b) receiving part of the probe signal, called a backscattered signal, that is scattered and reflected by the body and measuring the path length d of the backscattered signal;
(c) measuring a reflectance R of the part of the body traversed by the backscattered signal; and
(d) computing the attenuation coefficient μ based on the measured path length d and on the reflectance R.

2. The method according to claim 1, the attenuation coefficient μ being computed based on the following formula:
$$R = \frac{{e}^{- μ d}}{{d}^{2}} .$$

3. The method according to claim 1, the measurement of the reflectance R comprising measuring an average intensity iᵣ of the backscattered signal and computing the reflectance R using the following formula:
$$R = \frac{{i}_{r}}{{i}_{e}}$$
where iₑ is the average intensity of the probe signal irradiating the body.

4. The method according to claim 1, the measurement of the reflectance R comprising transmitting an additional probe signal having a same wavelength as the optical radiation and irradiating the body, followed by measuring an average intensity iᵣ′ of part of the additional probe signal, called an additional backscattered signal, scattered and reflected by the body and then computing the reflectance R using the following formula:
$$R = \frac{{i}_{r}^{′}}{{i}_{e}^{′}}$$
where iₑ′ is the average intensity of the additional probe signal irradiating the body.

5. The method according to claim 4, the additional probe signal having a constant intensity.

6. The method according to claim 1, wherein step (a) further comprises modulating the optical frequency of the optical radiation over a ramp of height h and duration t, and splitting the optical radiation into the probe signal and into a local oscillator, with the local oscillator not irradiating the body, and
the measurement of the reflectance R further comprises mixing the backscattered signal and the local oscillator in order to form an electromagnetic beat, followed by computing the reflectance R based on the modulation amplitude of the intensity of the electromagnetic beat and/or based on a value of the direct part of the intensity of the electromagnetic beat.

7. The method according to claim 1, the optical radiation being sinusoidally intensity-modulated.

8. The method according to claim 7, the measurement of the path length d further comprising measuring a phase shift α between the backscattered signal and the emitted optical radiation, followed by computing the path length d based on the following formula:
$$d = c \cdot \frac{α}{4 \cdot π \cdot {f}_{m o d}}$$
where c is the speed of light in a vacuum and f_{mod} is a modulation frequency of the optical radiation.

9. The method according to claim 1, for measuring the path length d, wherein step (a) further comprises modulating the optical frequency of the optical radiation over a ramp of height h and duration t;
step (a) further comprises splitting the optical radiation into the probe signal and into a local oscillator, with the local oscillator not irradiating the body; and
step (b) further comprises mixing the backscattered signal and the local oscillator in order to form an electromagnetic beat, followed by computing the path length d based on the following formula:
$${f}_{beat} = h \cdot \frac{d}{c \cdot t}$$
where f_{beat} is an average frequency of the electromagnetic beat.

10. The method according to claim 9, the frequency f_{beat} of the electromagnetic beat being obtained by processing comprising a Fourier transform of an intensity of the electromagnetic beat.

11. A device for implementing the method according to claim 8, the device comprising:
a light emission source configured to emit sinusoidally intensity-modulated optical radiation and at least part of which, called the probe signal, is intended to irradiate a scattering and/or absorbing body;
an optical detector configured to measure the phase shift α between part of the probe signal, called the backscattered signal, scattered and reflected by the body and the emitted optical radiation, and to measure the average intensity of the backscattered signal, with the light emission source and the optical detector being designed to be disposed on the same side of the body; and
a data processing unit configured to compute the path length d based on the following formula:
$$d = c \cdot \frac{α}{4 \cdot π \cdot {f}_{m o d}}$$
where c is the speed of light in a vacuum and f_{mod} is the modulation frequency of the optical radiation; and to compute the attenuation coefficient μ based on the computed path length d and on the reflectance R of the part of the body traversed by the backscattered signal measured based on the average intensity measured by the optical detector.

12. The device according to claim 11, the optical detector comprising at least one current-assisted photonic demodulator.

13. The device according to claim 11, the optical detector comprising at least one PIN junction photodiode.

14. A device for implementing the method according to claim 9, the device comprising:
a light emission source configured to emit coherent, intensity-modulated optical radiation with an optical frequency that is modulated over a ramp of height h and duration t;
a beam splitter configured to split the optical radiation into a probe signal for irradiating a scattering and/or absorbing body and a local oscillator;
an optical detection unit comprising a multiplexer for mixing the local oscillator with part of the probe signal, called the backscattered signal, scattered and reflected by the body in order to form an electromagnetic beat, with the optical detection unit also comprising at least one optical receiver configured to measure the intensity of the electromagnetic beat over time or also configured to measure the average intensity of the backscattered signal, with the light emission source, the beam splitter and the optical detection unit being designed to be disposed on the same side of the body; and
a data processing unit configured to determine the average frequency f_{beat} of the electromagnetic beat based on its measured intensity and to compute the path length d based on the following formula:
$${f}_{beat} = h \cdot \frac{d}{c \cdot t}$$
and to compute the attenuation coefficient μ based on the computed path length d and on the reflectance R of the part of the body traversed by the backscattered signal measured based on the average intensity of the backscattered signal measured by the optical receiver or based on the intensity of the electromagnetic beat.

15. The device according to claim 14, further comprising a waveguide (70) for guiding the local oscillator from the beam splitter to the optical detection unit.

16. The device according to claim 14, wherein the optical receiver comprises at least one PIN junction photodiode.

17. The device according to claim 11, wherein the data processing unit is further configured to perform the computation of the attenuation coefficient μ based on the following formula:
$$R = \frac{{e}^{- μ d}}{{d}^{2}} .$$

18. The device according to claim 11, the light emission source being further configured to emit an additional probe signal with the same wavelength as the optical radiation, with a constant intensity and that is intended to irradiate the body.
